(19) United States
(12) Patent Application Publication
(10) Pub. No.: US 2022/0356433 A1
(43) Pub. Date: Nov. 10, 2022

(54) MICROPHYSIOLOGICAL 3-D PRINTING AND ITS APPLICATIONS

(71) Applicant: Lung Biotechnology PBC, Silver Spring, MD (US)

(72) Inventors: Masoud Modaresifar, Manchester, NH (US); Mohammadali Safavieh, Nashua, NH (US); Keerthana Prakash, Boston, MA (US); Greg Hurst, Bedford, NH (US); Daniel E. Backman, Bedford, NH (US); Derek Morris, Bedford, NH (US); Akbar Khalilpour, Silver Spring, MD (US); Rebecca Duffy, Silver Spring, MD (US); Aman Kaur, Manchester, NH (US); Isabel Arias, Manchester, NH (US); Barbara Nsiah, Manchester, NH (US); Luis Alvarez, Lexington, MA (US)

(73) Assignee: Lung Biotechnology PBC, Silver Spring, MD (US)

(21) Appl. No.: 17/738,833

(22) Filed: May 6, 2022

Related U.S. Application Data
(60) Provisional application No. 63/185,298, filed on May 6, 2021.

(51) Int. Cl. C12M 1/12; C12N 5/071; C12N 5/00; C12M 3/06; C12M 1/00; B01L 3/00; A61L 27/38

(52) U.S. Cl. CPC C12M 25/14; C12N 5/069; C12N 5/0697; C12N 5/0062; C12M 23/16; C12M 29/10; B01L 3/502715; A61L 27/3808; B33Y 70/00

(57) ABSTRACT

The systems and methods of the present disclosure can be used to generate systems and models that are physiologically relevant to the human and animal system. These physiological conditions can be designed to mimic the actual human condition for cell differentiation and proliferation. The system and methods of this present disclosure allow the formation of an appropriate biomaterial to mimic that which exists in a human or animal scaffold. Utilizing 3D printing technology, a hydrogel scaffold can be printed at various resolution very close to human physiological geometry. Additionally, the architecture can be optimized for the selected application and appropriate cells can be seeded on the scaffold prior to testing.

### CROSS-REFERENCE TO RELATED APPLICATIONS

This application claims priority to U.S. Provisional Application No. 63/185,298, filed May 6, 2021, the entire contents of which are incorporated herein by reference.

### TECHNICAL FIELD

The present application relates to processes to present a micro-physiological 3D printed scaffold to mimic physiological human conditions to study various cells and micro-scaffolds at near close physiological resolution or evaluate its gas exchange and oxygen/CO₂ transfer between two separate complex structure in a 3D printed scaffolds.

### BACKGROUND

3D cell culture models may be used to study human and animal physiological conditions.

### SUMMARY

One embodiment is to provide a 3D printed unit that can monitor bio-scaffold, cell interface study using microscopy imaging.

Another embodiment is to evaluate various 3D cell culturing where geometry and structure of 3D printed scaffold mimic physiological environment.

Another embodiment is a 3D printed micro physiological unit to evaluate various chemical components and drugs efficacy on specific cell types with a vasculature network.

Another embodiment is to provide complex 3D printed vasculature model to create vasculature systems consisting of one or several human cell types.

Another embodiment is a gas exchange unit comprising a vascular network configured to conduct blood and an airway compartment configured to hold air comprising oxygen, wherein the vascular network contacts the airway compartment to permit gas exchange and increase oxygen content of blood passing through the vascular network. Another embodiment is an artificial lung comprising the gas exchange unit.

Another embodiment is a method of forming a gas exchange unit, comprising printing a gas exchange unit comprising a vascular network configured to conduct blood and an airway compartment configured to hold air comprising oxygen wherein the vascular network contacts the airway compartment to permit gas exchange and increase oxygen content of blood passing through the vascular network.

Another embodiment is a system-on-a-chip device comprising a gas exchange unit comprising a vascular network configured to conduct blood and an airway compartment configured to hold air comprising oxygen, wherein the vascular network contacts the airway compartment to permit gas exchange and increase oxygen content of blood passing through the vascular network seeded with cells.

### BRIEF DESCRIPTION OF THE DRAWINGS

FIGS. 1A-1B illustrates a 3D printed lung on a chip platform. FIG. 1A illustrates a schematic of alveolar gas exchange units. FIG. 1B illustrates a 3D printed scaffold perfused with blood inside vascular and air.

FIG. 2 illustrates a schematic of an inverted digital light projection (DLP) system.

FIGS. 3A-3C illustrate the placement of the 3-D printed microfluidic on the 3-D printer for 3-D printing the internal lung-on-a-chip according to one embodiment.

FIGS. 4A-4D illustrate a schematic of 3-D printed microfluidic parts according to one embodiment. FIG. 4A illustrates the Inlet dispenser according to one embodiment. FIG. 4B illustrates the 3D printed hydrogel according to one embodiment. FIG. 4C illustrates the 3D printed plastic container which will hold the 3d printed gel according to one embodiment. FIG. 4D illustrated the outlet dispenser according to one embodiment.

FIGS. 5A-5D illustrate images of the fluidic components for the two lumen design in different sizes according to some embodiments. FIG. 5A illustrates three diffent embodiment of the fluidic design before the hydrogel is 3-D printed according to some embodiments. FIG. 5B, FIG. 5C and FIG. 5D are embodiments of the assembly including the 3D printed microfluidic with a 3D printed hydrogel perfused inside.

FIGS. 6A-6D illustrate embodiments of various architectures of the micro-physiological unit. FIG. 6A illustrates a capsule net architecture. FIG. 6B illustrates a giant Fischer architecture. FIG. 6C illustrates a Fischer block architecture. FIG. 6D illustrates a cubic net architecture.

FIGS. 7A-7D illustrates embodiments of the systems and methods of the application. FIG. 7A shows different architectures of fluidic design for the 3-D printed vasculature. FIG. 7B show images of cells seeded on the formed vasculature and airway. FIG. 7C is a photograph of a microscopic setup for imaging. FIG. 7D is an example of a test setup where blood is perfused in the formed vasculature and gas is perfused in the formed airway.

FIG. 8 illustrates 3-D printed hydrogel gas exchange unit designs with different dimensions according to some embodiments.

FIG. 9 illustrates 3-D printed lung-on-a-chip designs made with different bioink formulations.

FIG. 10 illustrates a 2D gas exchange membrane chip and a plot of gas exchange data according to one embodiment,

FIG. 11 illustrates a microphysiological model perfused with whole blood according to some embodiments.

FIGS. 12A-12C illustrate a lung on a chip platform according to one embodiment. FIG. 12A illustrates a schematic of alveolar gas exchange units according to one embodiment. FIG. 12B illustrates a 3D printed scaffold perfused with blood inside vasculature and air according to one embodiment. FIG. 12C shows a plot of data of the gas exchange across the 3D printed hydrogel.

FIG. 13 illustrates different 3D printed hydrogel gas exchange unit designs with a plot of the increase in oxygen content measured from the outlet compared with the inlet.

FIGS. 14A-14F illustrate a 2D Gas exchange membrane chip according to one embodiment. FIG.14A illustrates a membrane based gas exchange unit according to some embodiment. FIG. 14B illustrates the membrane based gas exchange until with the perfusion of blood inside. FIGS. 14C-14F illustrate plots of gas exchange data for a collagen membrane and PDMS membrane.

FIG. 15 illustrates Endothelial cell seeding in this hydrogel using 3D printed Gel matrix and a variety of cellularization conditions.

FIGS. 16A-16C illustrate an acellular and cellular gas exchange assay according to some embodiments. FIG. 16A illustrates a schematic of the capsule net model according to some embodiments. FIG. 16B illustrates whole human blood perfused at low oxygen into the cube net model. FIG. 16C illustrates the gas exchange rate achieved for the acellular and cellular gas exchange assays compared with a control.

FIG. 17 is an image of the holder designed for Example 1, according to some embodiments.

FIGS. 18A-18B, 19A-19B, 20A-20B, 21A-21B, 22A-22B, and 23A-23B are embodiments according to Example 2, according to some embodiments.

Like reference numbers and designations in the various drawings indicate like elements.

### DETAILED DESCRIPTION

Unless otherwise specified, “a” or “an” means “one or more.”

The present application incorporates by reference in their entirety each of the following documents: (a) U.S. provisional application No. 63/185293 filed May 6, 2021 titled “USE OF FUNCTIONALIZED AND NON-FUNCTIONALIZED ECMS, ECM FRAGMENTS, PEPTIDES AND BIOACTIVE COMPONENTS TO CREATE CELL ADHESIVE 3D PRINTED OBJECTS” and U.S. non-provisional and/or PCT application(s) under the same title filed on May 6, 2022; (b) U.S. provisional application No. 63/185302 filed May 6, 2021 titled “MODIFIED 3D-PRINTED OBJECTS AND THEIR USES” and U.S. non-provisional and/or PCT application(s) under the same title filed on May 6, 2022; (c) U.S. provisional application No. 63/185305 filed May 6, 2021 titled “PHOTOCURABLE REINFORCEMENT OF 3D PRINTED HYDROGEL OBJECTS” and U.S. non-provisional and/or PCT application(s) under the same title filed on May 6, 2022; (d) U.S. provisional application No. 63/185299 filed May 6, 2021 titled “ADDITIVE MANUFACTURING OF HYDROGEL TUBES FOR BIOMEDICAL APPLICATIONS” and U.S. non-provisional and/or PCT application(s) under the same title filed on May 6, 2022; (e) U.S. provisional application No. 63/185300 filed May 6, 2021 titled “CONTROLLING THE SIZE OF 3D PRINTING HYDROGEL OBJECTS USING HYDROPHILIC MONOMERS, HYDROPHOBIC MONOMERS, AND CROSSLINKERS” and U.S. non-provisional and/or PCT application(s) under the same title filed on May 6, 2022.

The systems and methods of the present disclosure can be used to generate systems and models that are physiologically relevant to the human and animal system, including disease state models. These physiological conditions can be designed to mimic the actual human condition for cell differentiation and proliferation. The system and methods of this present disclosure allow the formation of a scaffold that mimics a biological scaffold, e.g., the extracellular matrix (ECM) of a human lung, using a material, such as a hydrogel or other polymer. A polymer scaffold can be printed using 3D printing techniques at various resolution, such as a resolution close to human physiological geometry. The architecture can be optimized for the selected application, and appropriate cells can be seeded on the scaffold prior to testing.

Microphysiological modela can be used as a surrogate for animal or human testing, for example, and can permit more efficient, cheaper, and or faster testing. The systems and methods described herein can be used to study a variety of physiological processes, such as the effects of potential therapeutics and cell expansion and differentiation. For example, the systems described herein can be used to model normal or altered, e.g., diseased or damaged, states. These models can be used to evaluate potential therapeutics or cellular or other physiological responses in these states. Additionally, microphysiological model designs have the potential for allowing the generation of synthetic organs for the treatment of disease. Unfortunately, there are difficulties in designing microphysiological model to mimic native physiological dimensions due, in part, to manufacturing difficulties.

The systems and methods in this disclosure allow the generation of large number of variations of microphysiological model designs. In some embodiments, these microphysiological models may be a lung-on-a-chip design. The system and methods disclosed allow for the manufacture of technically challenging, but physiologically relevant, aspect ratios. The formed lung-on-a-chip may be of various architectures, and these architectures may be tested to optimize the use. Described herein are various architectures and embodiments, however, these should not be considered limiting as they are merely examples of the architectures designed and tested for the particular use cases selected for that particular embodiment.

Additionally, 3D cell culture models have gained interest due to potential of providing physiologically relevant conditions for study and application. These physiological conditions can be designed to mimic the actual human condition for cell differentiation and proliferation. Unfortunately, these current modeling platforms utilize synthetic polymers such as poly dimethyl siloxhane (PDMS), which are unlike natural conditions.

In contrast, the system and methods of this present disclosure allow the formation of an appropriate biomaterial to mimic that which exists in a human or animal scaffold. Utilizing 3D printing technology, a hydrogel scaffold can be printed at various resolutions, including resolutions close to or at human physiological geometry. This scaffold may be formed using natural polymers such as Collagen type I or Gelatine. Using such biomaterials, the scaffold provides very close material properties to those of a native human scaffold and allows the proliferation of various types of cells.

This disclosure addresses systems and methods of making and using a 3D printed hydrogel that can mimic a human scaffold. This scaffold may be made from natural hydrogel. These systems and methods may be used as a testing platform to evaluate different bioinks and hydrogel scaffold on the proliferation of different cell types, drug screening in 3D culture environment, drug screening, drug efficacy on different cell types, pharmacokinetics and pharmacodynamic studies. Additionally, these systems and methods may be used for 3D printing a scaffold which may be used for tissue repair.

This microphysiological system also provides a gas exchange, as described in greater detail below. The gas exchange unit can comprise an airway compartment and a vascular network. A variety of parameters for the airway compartment and vascular network can be customized depending on application: airway volume, airway surface area, vasculature volume, vasculature area, vascular lumen diameter, airway vascular interface thickness, and airway vascular orientation. The airway compartment and vascular network can be made from a biomaterial, such as a hydrogel or other polymer with or without additional components. The airway compartment and vascular network can be 3D printed with any printable bioink to form a hydrogel. Cells can be seeded, cultured, and perfused as part of the airway compartment and vascular network. Various configurations and adaptations of the gas exchange unit are described in greater detail below.

The gas exchange unit may include a vascular network configured to conduct a fluid, such as blood or a blood substitute (e.g., a perfluorocarbon blood substitute). The gas unit may include an airway compartment configured to hold a gas. The gas can be some combination of gases, such as air, and can comprise oxygen. The vascular network may contact the airway compartment to permit gas exchange between the fluid in the vascular network and the gas in the airway compartment. In some embodiments, the gas exchange increases oxygen content of the fluid. In some embodiments, the fluid may release carbon dioxide into the airway compartment. The gas exchange unit may be seeded with any suitable cell type, including pulmonary artery endothelial cells. The gas exchange unit composition may include a hydrogel. The gas exchange unit composition may include one or more compounds such as polyethylene glycol, polyethylene glycol diacrylate, polyethylene glycol methacrylate, polyethyleneglycolmethylether, N, N′-methylenebiasacrylamide and methacrylated collagen.

The gas exchange unit may have an interface between the vascular network and an airway compartment. In some embodiments, the diameter of an interface between the vascular network and the airway compartment is between 250 microns and 350 microns. The vascular network may include a lumen. In some embodiments, the lumen of the vascular network may be between 350 microns and 450 microns. In some embodiments, the diameter of the lumen of the vascular network may be between 150 microns and 250 microns. The diameter of the lumen of the vascular network may be greater than the diameter of the interface between the vascular network and the airway compartment

The microphysiological unit that provides gas exchange may be constructed to have any of a variety of architectures. These architectures may be modeled off of biological organs such as lungs, kidneys, hearts, intestines, or other organs. These architectures may be modeled based on the underlying principle to maximize surface to volume ratio of construct comprising vasculature and airway networks.

The gas exchange unit may be fabricated using biomaterials or other materials that mimic a human or animal scaffold. The gas exchange unit may include a biomaterial hydrogel scaffold. The biomaterial hydrogel scaffold may include a natural polymer. The natural polymer may be one or more of Collagen and Gelatin. The natural polymer may be Gelatin.

The gas exchange unit may be seeded with cells. In some embodiments, the cells may be endothelial cells. The gas exchange unit may be seeded with small airway epithelial cells (SAEC) on one side of the biomaterial hydrogel scaffold. The gas exchange unit may be seeded with endothelial cells on the other side of the biomaterial hydrogel scaffold.

The method may include measuring the gas exchange between the vascular network and the airway compartment. Measuring the gas exchange between the vascular network and the airway compartment can be used as a metric for monitoring cell growth, expansion, or differentiation. In some embodiments, oxygen exchanged between the airway compartment and a fluid in the vascular network can be monitored. In some embodiments, carbon dioxide exchange between the fluid in the vascular network and airway compartment can be monitored.

Another aspect of the present disclosure is directed to an artificial lung comprising the gas exchange unit. The artificial lung can comprise a plurality of gas exchange units arranged in any suitable geometry. For example, a single vascular network can be in contact with a plurality of airway compartments. The gas exchange units can be arranged serially or in parallel. The gas exchange units may be seeded with one or more cell types to mimic one or more physiological conditions.

Another aspect of the present disclosure is directed to a method of forming a gas exchange unit. The method may include printing a gas exchange unit using, for example, one or more 3D printing techniques. The gas exchange unit may include a vascular network configured to conduct blood and an airway compartment configured to hold a gas or mixture of gases comprising oxygen, e.g., air. The vascular network may contact the airway compartment to permit gas exchange and increase oxygen content of blood passing through the vascular network. The gas exchange unit may be printed using a 3D printer. The gas exchange unit may be printed using a bioink. The gas exchange unit may be printed using an ink including one or more compounds selected from the group polyethylene glycol, polyethylene glycol diacrylate, polyethylene glycol methacrylate, Polyethyleneglycolmethylether, N, N′-Methylenebiasacrylamide and methacrylated collagen. The gas exchange unit may be printed using an ink including one or more compounds including methacrylated collagen, poly ethylene glycol diacrylate, lithium phenyl-2,4,6-trimethylbenzophosphinate, UV386A dye, and 3-Hydroxypropylacrylate. The bioink can be one or more of the bioinks described in co-pending application filed May 6, 2021 entitled “USE OF FUNCTIONALIZED AND NON-FUNCTIONALIZED ECMS, ECM FRAGMENTS, PEPTIDES AND BIOACTIVE COMPONENTS TO CREATE CELL ADHESIVE 3D PRINTED OBJECTS”, which is hereby incorporated by reference in its entirety for disclosure of bioinks.

The method may include seeding the gas exchange unit or vascular network with any suitable cells in one or more steps. In embodiments such as pulmonary bio, cells may include one or more of lung smooth muscle cells, lung fibroblasts, lung mesenchymal stem cells, induced pluriprotent stem cells, and cell derived cell types. In some embodiments, stems cells or other precursor cells are differentiated into suitable cells types after seeding the gas exchange unit with the cells. Gas can be provided to the gas exchange unit to facilitate cell seeding, expansion, differentiation, or otherwise mimic different physiological conditions. Likewise, a fluid, such as whole blood, can be perfused in the vascular network to facilitate cell seeding, expansion, differentiation, or otherwise mimic different physiological conditions. In some embodiments, the method comprises seeding the cells on the gas exchange unit and vascular network scaffolds simultaneously. In other embodiments, the cells can be seeded in stages, e.g., the airway compartment is seeded before the vascular network. The method can comprise providing growth factors, cytokines, or other components to facilitate cell seeding, expansion, differentiation, or otherwise mimic different physiological conditions. These components can be provided using gas in the airway compartment, fluid in the vascular network, or by other means.

Another aspect of the present disclosure is directed to a method of utilizing the system-on-a-chip device to provide physiologically relevant conditions for ex vivo models. The system-on-a-chip device may be used to screening pharmaceutical compositions. The system-on-a-chip device may be used to model pulmonary disorders, such as pulmonary hypertension in any of its forms, e.g., pulmonary arterial hypertension. The system-on-a-chip device may be used to perform pulmonary toxicity studies.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

The details of one or more implementations of the subject matter described in this specification are set forth in the accompanying drawings and the description below. Other features, aspects, and advantages of the subject matter will become apparent from the description, the drawings, and the claims.

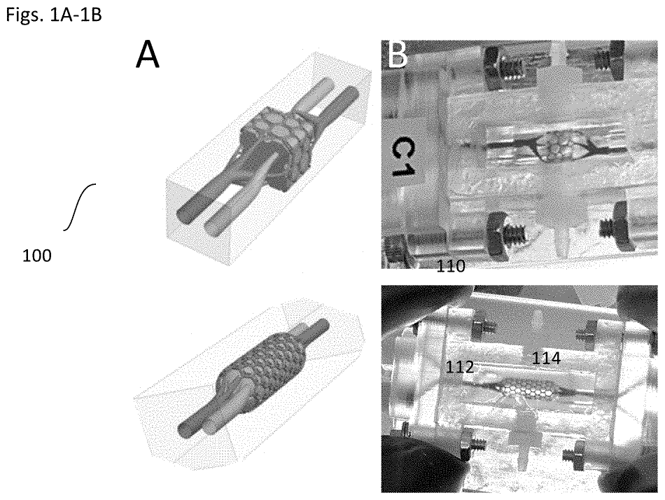

FIGS. 1A-1B illustrate a printed lung on a chip platform according to one embodiment. The printed lung on a chip platform may include one or more alveolar gas exchange units. FIG. 1A illustrates a schematic of alveolar gas exchange units 100 according to one embodiment. The alveolar gas exchange unit may be a hydrogel gas exchange unit 100. The gas exchange unit 100 may include an airway and vascular compartment. The gas exchange unit 100 may have the following customizable parameters: airway volume, airway surface area, vasculature volume, vasculature area, vascular lumen diameter, airway vascular interface thickness, and airway vascular orientation. The gas exchange unit 100 may be 3D printed be printed with any printable ink ranging from plastic resin to bioink, to hydrogel.

Within the formed gas exchange unit 100 cells can be seeded, cultured, and perfused. Within the gas exchange unit 100, whole blood can be perfused and gas exchange can be measured. This gas exchange unit 100 can enable evaluation of relevant cell types for lung tissue engineering, airway vascular designs for lung tissue engineering, and materials which meet mechanical, bioactive, and oxygen diffusion requirements for lung tissue engineering.

The alveolar gas exchange unit 100 may include a scaffold 110. The Alveolar gas exchange unit 100 may include a vascular network 112. The alveolar gas exchange unit 100 may include an air compartment 114. The lung on a chip platform and alveolar gas exchange unit 100 may be formed by 3D printing. The architecture of the microphysiological platform and the alveolar gas exchange unit 100 may vary according to the embodiment. FIG. 1B illustrates a scaffold 110 perfused with blood inside a vascular network 112 and air inside the air compartment 114. The scaffold may be formed by 3D printing. The scaffold may be a hydrogel. The hydrogel scaffold may be printed at various resolution very close to human physiological geometry according to some embodiments. The scaffold may be made of natural polymers. These natural polymers may be biomaterials such as Collagen, Gelatine, or other well-known biomaterials. Using such biomaterials, the scaffold may very close material properties to those of a native human scaffold.

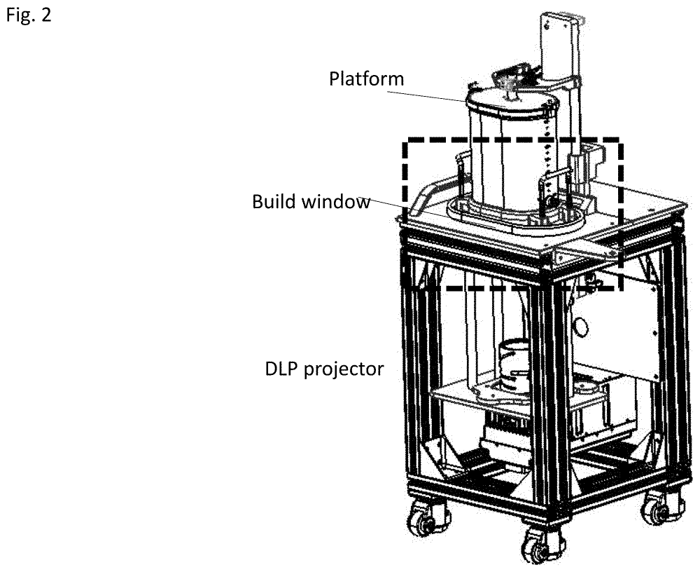

FIG. 2 illustrates a schematic of an inverted digital light projection (DLP) system 500. As an alternative to using a solid membrane with an inverted DLP 3D-printer. The system for forming a three-dimensional object can include a platform (e.g., print platform) on which the three-dimensional object is formed. The three-dimensional object can include an artificial organ (e.g., artificial lung, artificial heart, artificial kidney, artificial liver, etc.). The build surface and the platform can define a build region (e.g., build window) therebetween. The system can include a controller configured to advance the platform away from the build surface. For example, the controller can lower or raise the platform. The system can include a radiation source (e.g., DLP projector, projector, illumination source, etc.) configured to irradiate the build region. The radiation source can be configured to irradiate the build region through an optically transparent member to form a solid polymer from a photosensitive liquid (e.g., photosensitive resin, ink, etc.). Embodiments of the systems and methods used have been discussed in application 63/069317 filed Aug. 24, 2020 which is hereby incorporated by reference.

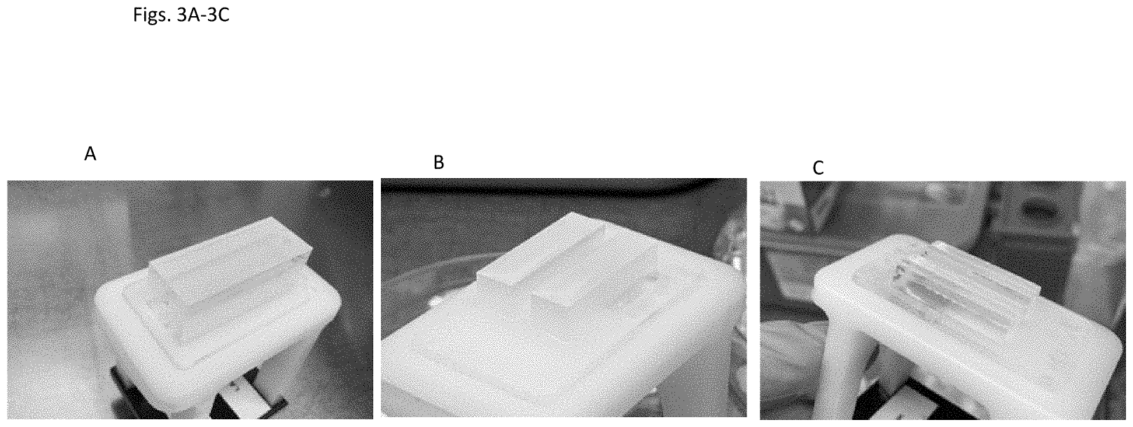

FIGS. 3A-3C illustrates a 3-D printed microfluidic parts according to one embodiment. FIG. 3A illustrates the inlet dispenser according to one embodiment. The inlet dispenser may be used to perfuse the gas exchange unit 100 with liquid or gas for testing or use. FIG. 3B illustrates the gas exchange unit 100 according to one embodiment. The gas exchange unit may be a hydrogel, polymer, or biomaterial printed into the microfluidic part. FIG. 3C illustrates the plastic container. The plastic container may be 3D printed. The plastic container may hold the gas exchange unit or hydrogel which may be 3D printed into the plastic container according to some embodiments. FIG. 3D illustrated the outlet dispenser according to one embodiment. In some embodiments. The outlet dispenser may be used to remove the liquid or gas perfused into the gas exchange unit 100. In some embodiments, the bottom and top of the microfluidic part may be covered with PDMS. It may prevent leakage of the printed hydrogel and enable drying. In some cases, the bottom and top will be covered using 134 um PDMS. The platform can include a flexible membrane. The membrane can include a polytetrafluoroethylene membrane. The membrane 702 can have a build surface where the 3D printed hydrogel can be placed. The build surface and the platform can have the build region there between.

The platform can include the ink (e.g., photosensitive ink). The photosensitive liquid can be disposed on the oxygen permeable membrane. The platform can include the radiation source. The radiation source can be configured to irradiate the build region 504 through an optically transparent member, and the oxygen permeable membrane to form a solid polymer from a photosensitive liquid.

The micro-physiological unit may be 3-d printed using DLP or SLA technique with photosensitive ink. The micro-physiological unit may be removed from the 3D printer and placed in a holder to further seed cells and evaluate the gas exchange between vasculature network and airways.

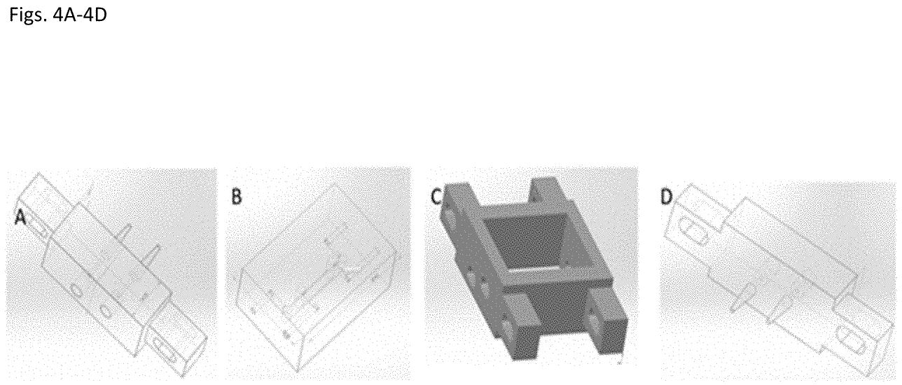

FIGS. 4A-4D illustrates images of the fluidic components and embodiments for the two lumen design in different microfluidic dimensions ranging from milimeter size channels down to 10 um channels. FIG. 4A illustrates three diffent embodiment of the fluidic design before the hydrogel is 3-D printed according to some embodiments. FIG. 4B, FIG. 4C and FIG. 4D are embodiments of the assembly including the 3D printed microfluidic with a 3D printed hydrogel perfused inside. The microfluidic dimensions may be optimized based on the desired application.

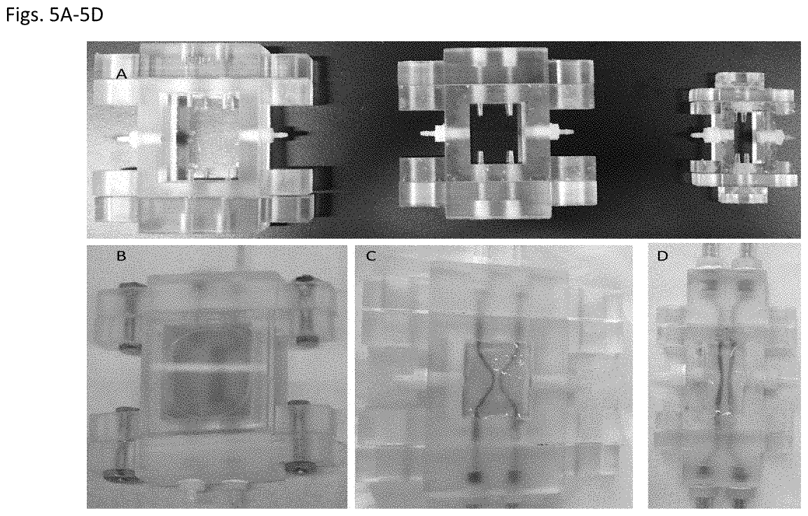

FIGS. 5A-5D illustrate the gas exchange unit designed with different dimensions according to some embodiments. The dimensions of the gas unit may be selected on the basis of the desired applications or the microphysiological dimensions. The dimensions may be selected to mimic physiologically relevant aspect ratios. The formed gas exchange unit may be of various architectures, and these architectures may be tested to optimize the use.

The gas exchange unit may include a vascular network configured to conduct blood. The gas unit may include an airway compartment configured to hold air comprising oxygen. The vascular network may contact the airway compartment to permit gas exchange and increase oxygen content of blood passing through the vascular network. The gas exchange unit may have an interface between the vascular network and an airway compartment. The diameter of an interface between the vascular network and the airway compartment is between 250 microns and 350 microns. The vascular network may include a lumen. The lumen of the vascular network may be between 350 microns and 450 microns. The diameter of the lumen of the vascular network may be between 150 microns and 250 microns. The diameter of the lumen of the vascular network may be greater than the diameter of the interface between the vascular network and the airway compartment. These dimensions are meant to be merely examples of the myriad of dimensions possible for a gas exchange unit and one skilled in the art would recognize the many alternatives that may be used.

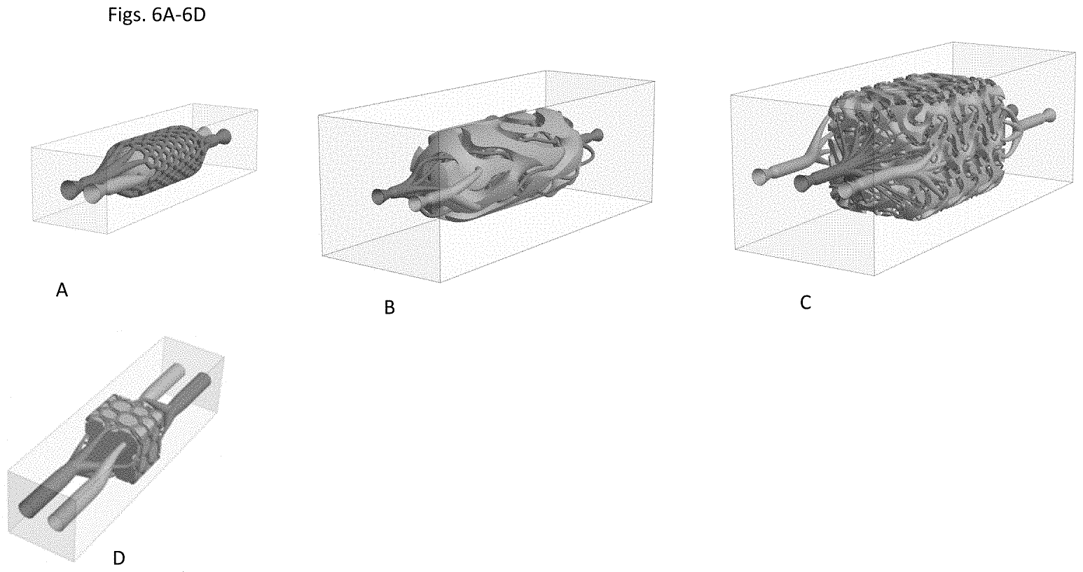

The microphysiological unit may be formed in various architectures. FIGS. 6A-6D shows an example of some of these architectures. FIG. 6A shows one embodiment where the architecture is a capsule net. A capsule net may be defined as a complex network of vasculature that surrounds a capsule-like cavity mimicking enlarged alveoli structure. FIG. 6B shows one embodiment where the architecture is a giant Fischer. A giant Fischer may be defined as a complex Fischer geometry with dense and complex vasculature within. FIG. 6A shows one embodiment where the architecture is a Fischer block. A fisher block may be defined as denser vasculature architecture in a Fischer foam. FIG. 6D shows one embodiment wherein the gas exchange unit has an architecture that is a cubic net. A cubic net may be defined as small cube cavity with vasculature network around it. These architectures are meant to be merely examples of the myriad of architectures possible to evaluate cell seeding in a complex structure and study the 3D printed scaffold interaction with various cell types as well as evaluate the gas exchange

The micro-physiological unit may include a vascular network. The vascular network may be seeded with endothelial cells. The vascular unit may be configured to conduct blood. The micro-physiological unit may include an airway compartment. The airway compartment may be seeded with epithelial cells or other cells such as Small Airway Epithelial Cells (SAEC). The airway compartment may be configured to hold air including oxygen. The vascular network may contact the airway compartment to permit gas exchange and increase oxygen content of blood passing through the vascular network seeded with cells.

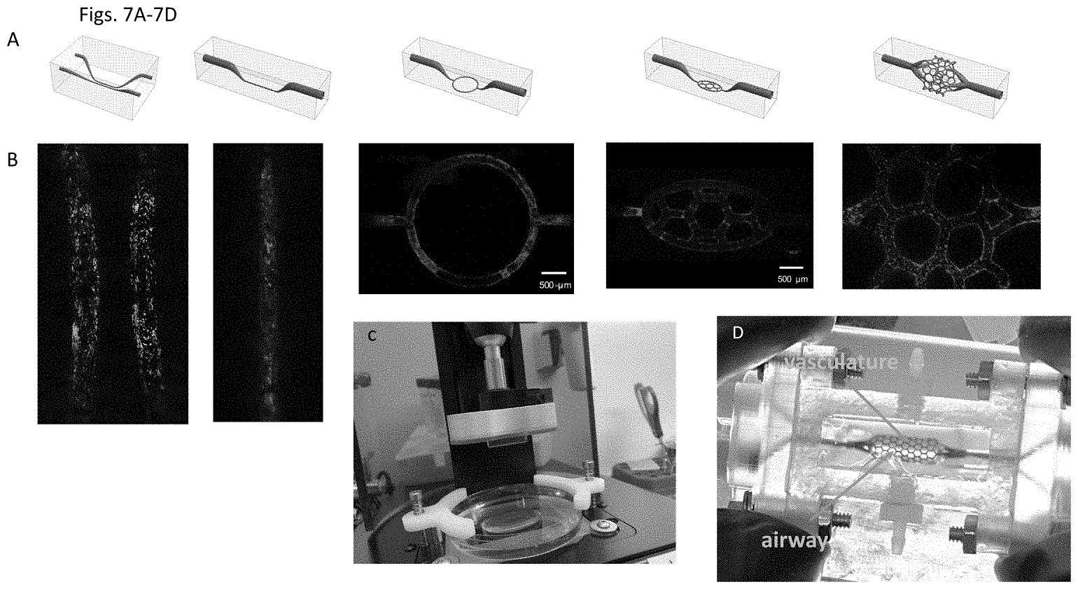

FIGS. 7A-7D illustrate embodiments of the systems and methods of the application. FIG. 7A shows different architectures of fluidic design for the 3-D printed vasculature. FIG. 7B show images of cells seeded on the formed vasculature and airway. FIG. 7C is a photograph of a microscopic setup for imaging. FIG. 7D is an example of a test setup where blood is perfused in the formed vasculature and gas is perfused in the formed airway.

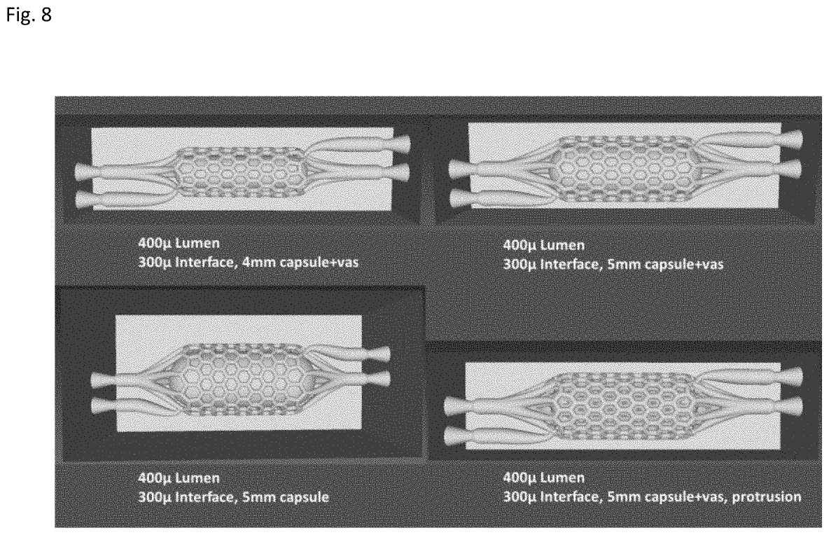

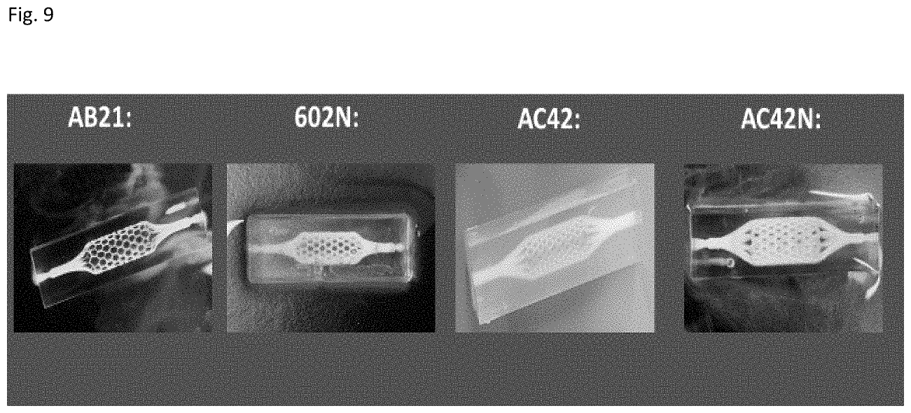

FIG. 8 illustrates lung-on-a-chip gas exchange unit designs made with a membrane at the interface between blood and air which was fabricated with different formulations. FIG. 9 illustrates 3-D printed lung-on-a-chip designs made with different bioink formulations.

The gas exchange unit may be 3-d printed using the methods disclosed above. The gas exchange unit may be printed using a bioink. The gas exchange unit may be printed using an ink including one or more compounds selected from the group polyethylene glycol, polyethylene glycol diacrylate, polyethylene glycol methacrylate, Polyethyleneglycolmethylether, N, N′-Methylenebiasacrylamide and methacrylated collagen. The gas exchange unit may be printed using an ink including one or more compounds including methacrylated collagen, poly ethylene glycol diacrylate, lithium phenyl-2,4,6-trimethylbenzophosphinate, UV386A dye, and 3-Hydroxypropylacrylate. This list is meant to be representative and those skilled in the art would recognize the wide variety of inks available now and in the future appropriate for use in 3-D printing.

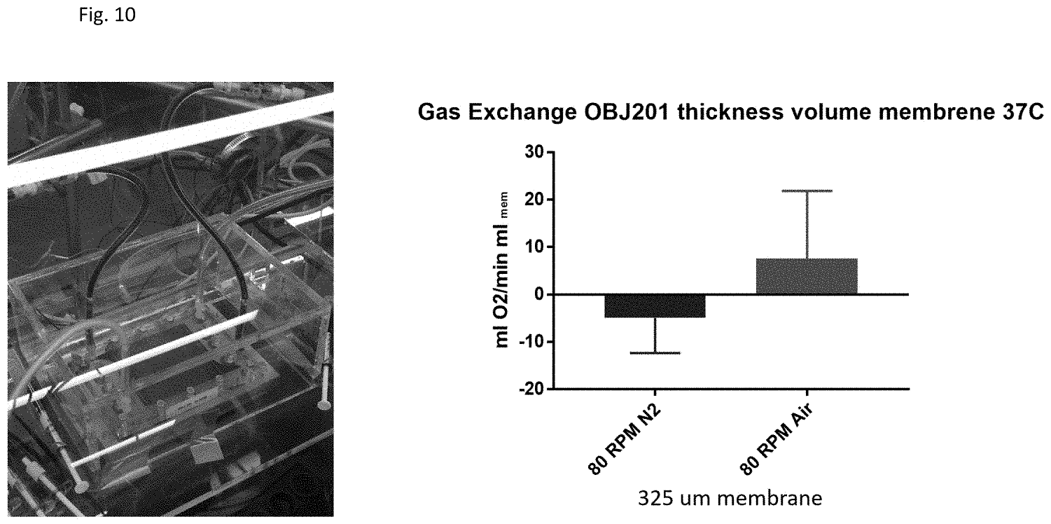

FIG. 10 illustrates a 2D gas exchange membrane chip and a plot of gas exchange data according to one embodiment. Once the gas exchange unit is formed, it can be tested to determine the amount of gas that is exchanged across the membrane. In this embodiment, a 325 um PDMS and bioink spin-coated membrane was used. The vasculature system gas exchange unit was perfused with blood. In this case, the change in concentration of oxygen in blood measured from the inlet of the gas exchange unit to the exit of the gas exchange unit is compared when 325 um membrane was used and Nitrogen as an inert gas is flowed and the result was compared when the air was flowed at 37° C. The gas exchange results in FIG. 7B shows that the gas exchange data is different using N2 and Air.

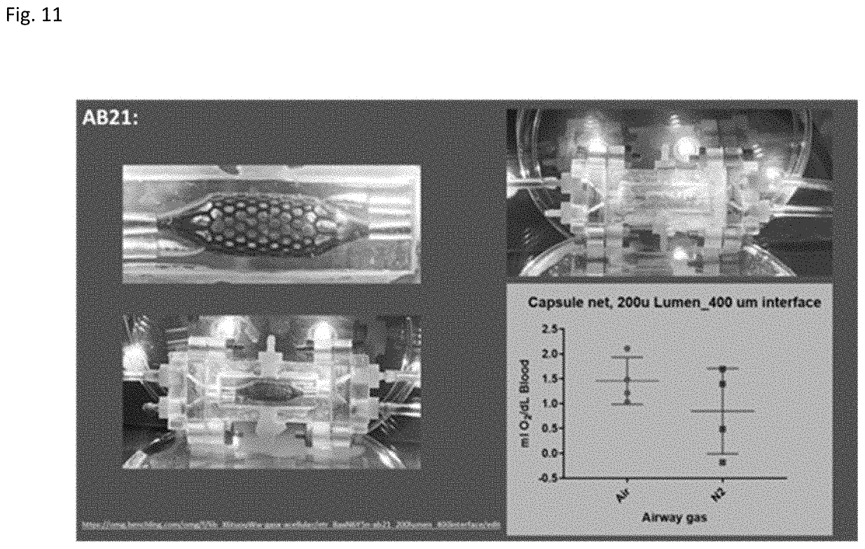

FIG. 11 illustrates a microphysiological model perfused with whole blood according to some embodiments. The microphysiological model may be formed using 3D printing. The microphysiological model may include a vascular compartment and an airway compartment. The vascular compartment and the airway compartment may be separated by a hydrogel wall. The lung on a chip model may be perfused with whole blood. Deoxygenated blood may be perfused into the inlet of the vascular compartment while air may be perfused into the airway compartment. The whole blood may travel through the vascular compartment. The whole blood may absorb oxygen as it passes through the vascular compartment through gas exchange across the membrane wall separating the airway compartment. The amount of oxygen absorbed by the blood traveling across the vascular compartment may be measured by comparing the concentration of oxygen in the blood between the inlet and outlet. This can be compared to change in the amount of oxygen in the blood when nitrogen is flowed through the airway compartment. A set of 4 measurements in a model component with cube-net architecture and a 200 um Lumen showed an average difference in measurement between the air test case and the nitrogen control.

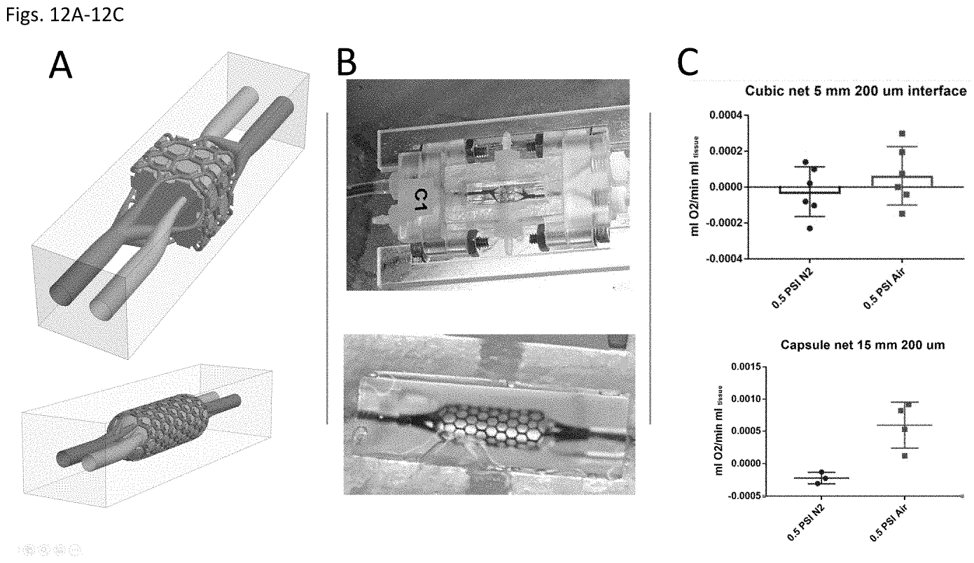

FIGS. 12A-12C illustrate a 3D printed lung on a chip platform according to one embodiment. FIG. 12A illustrates a schematic of alveolar gas exchange units according to one embodiment. This schematic shows the blue region of the airway compartment surrounded by the red region of the vascular compartment according to one embodiment. This schematic shows at the top a cubic net architecture and at the bottom capsule net architecture according to one embodiment which was printed using 5-15 (w/w)% PEGDA3400, 6-9 (w/w)% PEG575, 1-3 (w/w)% Lithium phenyl-2,4,6-trimethylbenzoylphosphinate and 0.13 (w/w)% UV386A. FIG. 12B illustrates a 3D printed scaffold perfused with blood inside vasculature and air according to one embodiment. The top is an image of the cubic net architecture with 5 mm vascular diameter and 200 um interface perfused with blood, according to one embodiment. The bottom is an image of the capsule net architecture with 15 mm vascular diameter and 200 um interface perfused with blood, according to one embodiment. The 3D printed scaffold was formed in the following manner: 5-15 (w/w)% PEGDA3400, 6-9 (w/w)% PEG575, 1-3 (w/w)% Lithium phenyl-2,4,6-trimethylbenzoylphosphinate and 0.13 (w/w)% UV386A. FIG. 12C shows a plot of data of the gas exchange across the 3D printed hydrogel from the gas exchange unit imaged in FIG. 8B. As it can be seen, the architecture and dimensions effect the amount of oxygen picked up by the blood as it flowed through the microphysiological device.

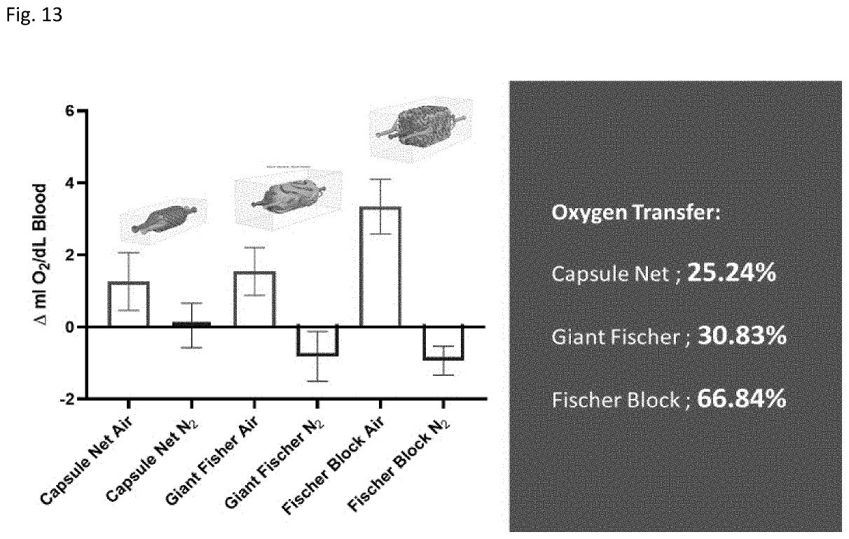

FIG. 13 illustrates different microphysiological unit designs with a plot of the increase in oxygen content of the blood measured at the outlet of the unit compared with the oxygen content of the blood measured at the inlet unit. Physiologically 100% oxygen transfer is defined as an increse from 15%-20% oxygen or a change of 5mL O₂/dL blood. The capsule net, giant fischer, and fischer block demonstrate microphysiological designs which have varyig levels of oxygen transfer and gas exchange capacitites. The lumen diameter of microphysiological units were varying from 200 um-500 um in vasculature. The interface between vasculature and airway was 400 um. The bioink was used for this work was 5% PEGDA 6000, 10% 4-HBA (4-Hydroxybutyl acrylate) 1.5% Lithium phenyl-2,4,6-trimethylbenzoylphosphinate and 0.1% UV386A. It is expectable that changing the diameter and interface of these microphysiological models yields different amount of gas exchange rate. As expected Fischer block that has highest surface area makes highest amount of oxygen transfer comparing with other scaffolds.

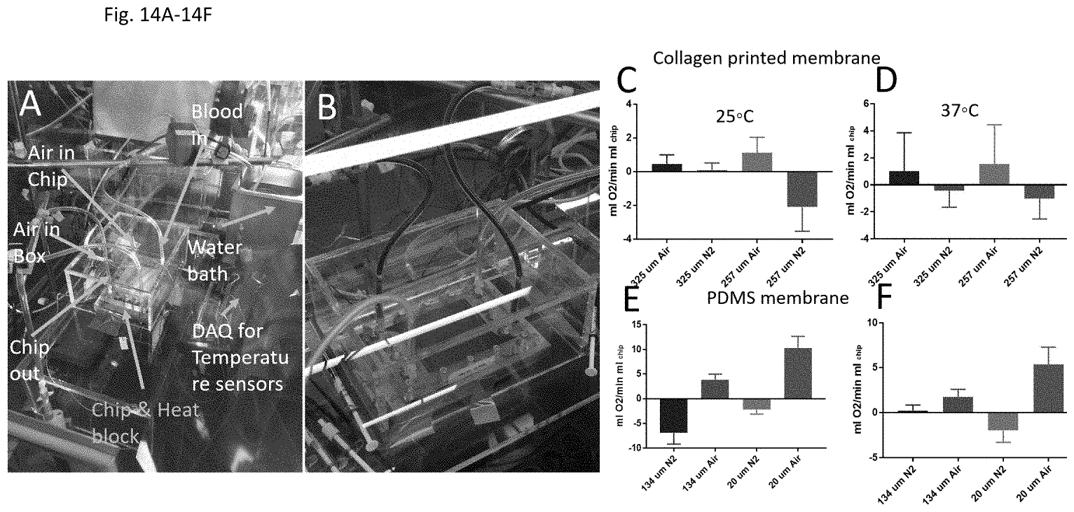

FIGS. 14A-14F illustrate a 2D lung on a chip made of different materials and measured at multiple temperatures according to one embodiment. FIG. 14A illustrates a membrane based gas exchange unit according to some embodiment. The unit is setup such that air can be infued into the air compartment and blood can be infused in the vascular compartment. The oxygen content of the blood can be measured at the inlet and the outlet of the vascular content. A water bath allows the test to be completed at multiple temperatures FIG. 14B illustrates the membrane based gas exchange unit with the perfusion of blood inside. FIG. 14C illustrates a plot of gas exchange data for a collagen membrane at 25° C. FIG. 14D illustrates a plot of gas exchange data for a collagen membrane at 37° C. FIG. 14E illustrates a plot of gas exchange data for a PDMS membrane at 25° C. FIG. 14F illustrates a plot of gas exchange data for a PDMS membrane at 37° C. The results shows that gas exchange rate is extensively lower at 37° C. comparing with the 25° C. This is falling with the physiological expectation that hemoglubin tends to loose oxygen at higher temperture yielding to have lower gas exchange rate.

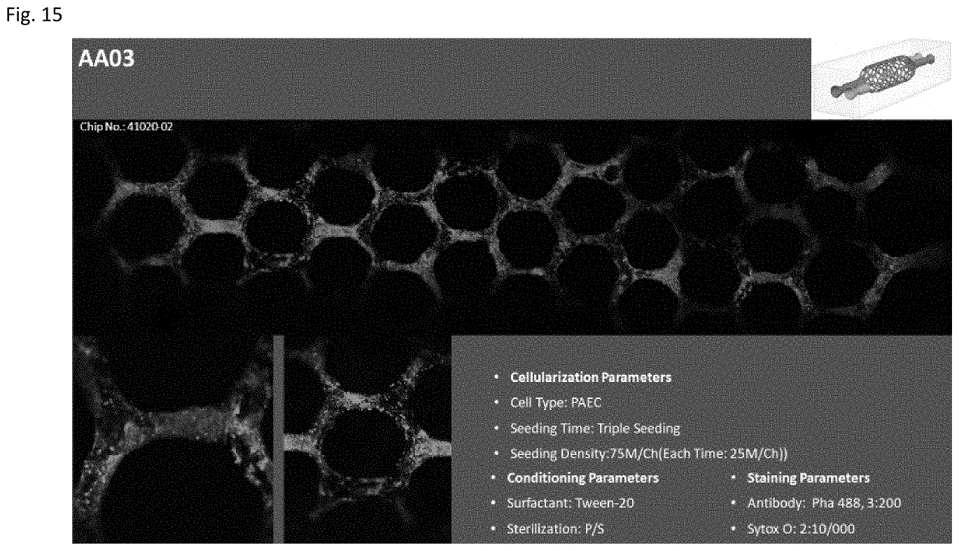

FIG. 15 illustrates Endothelial cell seeding in the microphysiological system under variety of cellularization conditions. The microphysiological scaffold was 3D printed using the methods discolsed above. The material was 8% PEGDA3400, 10% 3-Hydroxypicolinic acide (3-HPA), 1% Lithium phenyl-2,4,6-trimethylbenzoylphosphinate, and 0.1% UV386A. The platform was used to image various cellularization condition using different flow rates and cell densities seeded in different geometries. This illustration shows that the microphysiological system can be used to monitor endothelization procedure and interaction of endothelial cells on 3D printed hydrogel.

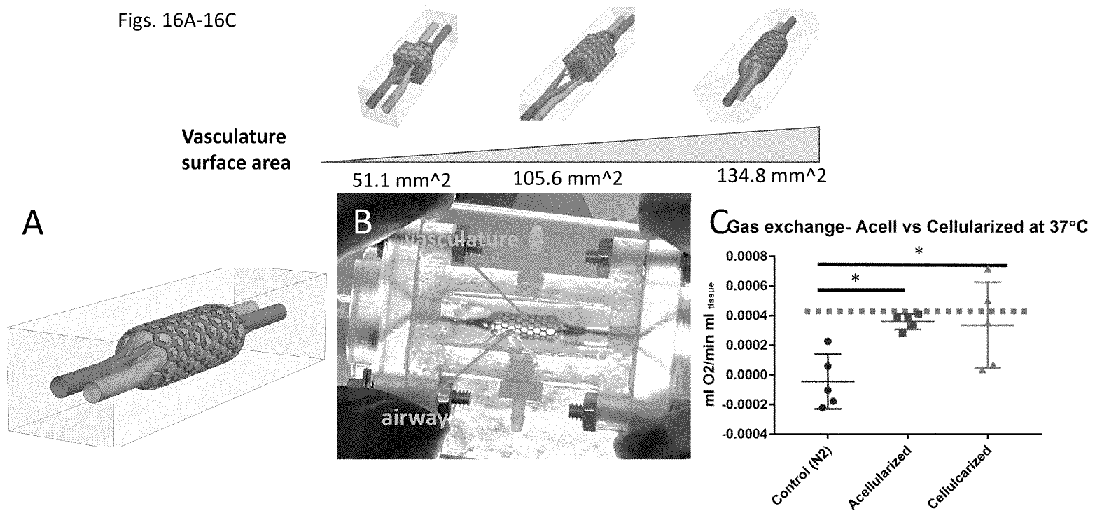

FIGS. 16A-16C illustrates an acellular and cellular gas exchange assay according to some embodiments. FIG. 16A illustrates a schematic of the capsule net model according to some embodiments. The capsule net was manufactured from was 5-15 (w/w)% PEGDA3400, 6-9 (w/w)% PEG575, 1-3 (w/w)% Lithium phenyl-2,4,6-trimethylbenzoylphosphinate and 0.13 (w/w)% UV386A. FIG. 16B illustrates whole human blood perfused at low oxygen into a cube net model. The material was 8% PEGDA2400, 10% -3Hydroxypicolinic acid (3-HPA), 1% Lithium phenyl-2,4,6-trimethylbenzoylphosphinate, 0.1% UV386A. FIG. 16C illustrates the gas exchange rate achieved for the acellular and cellular gas exchange assays compared with a control. The material was 8% PEGDA2400, 10% -3Hydroxypicolinic acid (3-HPA), 1% Lithium phenyl-2,4,6-trimethylbenzoylphosphinate, 0.1% UV386A. The architecture was 200 μm lumen. Endothelial cells were seeded with 100 μl min flow rate. The average of acellurlazed was 0.0004 ml O2/min ml tissue. The average of cellularized was 0.00038 ml O2/min mltissue. As can be seen above, the addtion of cells to the gas excahnge assay led no gas exchange difference between accellular and cellular assay.

The gas exchange unit may be seeded with cells of various types. The gas exchange unit may be seeded on the membrane. The seeding cells may be pulmonary artery endothelial cells. The cells may be endothelial cells. The cells may be epithelial cells. The cells may be small airway epithelial cells.

Different cells may be seeded on different sides of the gas exchange unit membrane. For instance, small airway epithelial cells (SAEC) are seeded on one side of the biomaterial hydrogel scaffold and endothelial cells are seeded on the other side of the biomaterial hydrogel scaffold. For instances, the SAEC may be seeded on the airway side of the biomaterial scaffold while endothelial cells may be seeded on the vascular side of the membrane.

The micro-physiological unit may be used as a physiological relevant model for multiple applications. For instance, in the case where the micro-physiological unit is a synthetic lung, pharmaceutical compositions may be screened for efficacy of drugs on Pulmonary disorders. Pulmonary toxicity studies may be performed on the system-on-a-chip device In other embodiments, the micro-physiological unit may be an alternate organ such as a kidney, liver, lung colon, heart or other organ. This list is meant to be merely exemplary and is not comprehensive. These synthetic organs may be used to screen for the toxicity or efficacy of drugs or other materials on the relevant system-on-a-chip device. Those in the field will recognize that many synthetic organs may be produced in this fashion and similar efficacy and toxicity studies may be made using the techniques described herein.

Any references to implementations or elements or acts of the systems and methods herein referred to in the singular can include implementations including a plurality of these elements, and any references in plural to any implementation or element or act herein can include implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

As utilized herein, the term “biomaterial” are intended to have a broad meaning in harmony with the common and accepted usage by those of ordinary skill in the art to which the subject matter of this disclosure pertains. Biomaterials may be natural and/or synthetic polymers. Biomaterials include other naturally occurring biological material as well as substances synthesized to mimic biological material. Such material may include polymers, hydrogels, peptides, proteins, cellulose, sugars, and various other materials known to those skilled in the art, whether derived from biological matter or synthetically formed.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

Any implementation disclosed herein may be combined with any other implementation, and references to “an implementation,” “some implementations,” “an alternate implementation,” “various implementations,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Elements other than ‘A’ and ‘B’ can also be included.

References herein to the positions of elements (e.g., “top,” “bottom,” “above,” “below”) are merely used to describe the orientation of various elements in the Figures. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The systems and methods described herein may be embodied in other specific forms without departing from the characteristics thereof. The foregoing implementations are illustrative rather than limiting of the described systems and methods.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

The following are Examples of the systems and methods disclosed herein. The following are merely examples and those skilled in the art will readily recognize the myriad of parameters that may be adapted using the systems and methods disclosed to optimize the systems and methods of the disclosure for various applications.

Example 1:

Bio-ink was formed by combining 5-15 (w/w)% PEGDA3400, 6-9 (w/w)% PEGDMA 575, 1-3 (w/w)% Lithium phenyl-2,4,6-trimethylbenzoylphosphinate and 0.13 (w/w)% UV386A by stepwise mixing PEGDA3400 in DI water andadding PEGDMA 575.. 1.5 (w/w)% Lithium phenyl-2,4,6-trimethylbenzoylphosphinate was added to the solution and mixed throughly. UV386A Dye was added to the solution and mixed. The bioink was placed in the vat of a 3D printer which was custom made by 3DSYSTEMS Corp.

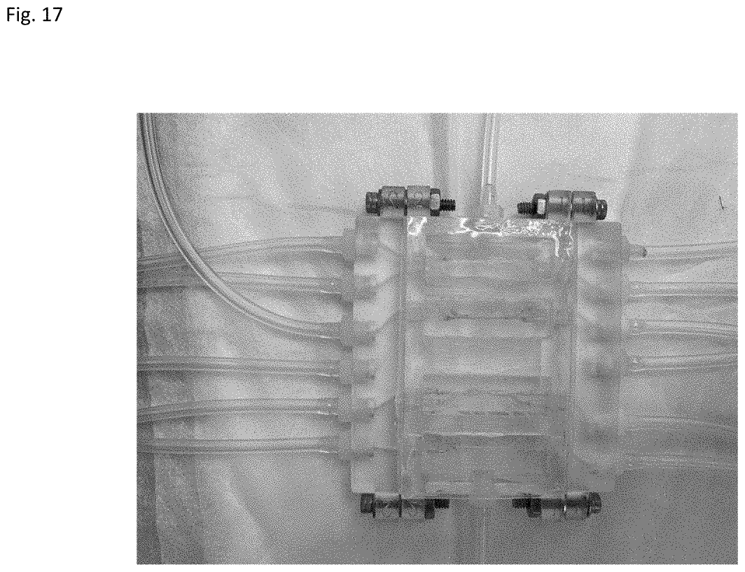

A custom designed 3-d printed microfluidic holder was used as shown in FIG. 17. The microfluidic holder was printed using commercially available plastic resin and a Formlab printer. The hydrogel microfluidic was printed in custom made 3DSYSTEMS bioprinter and placed into the microfluidic holder. Embodiments of the 3-d printed microfluidic were formed using various developed ink. As an example, in one embodiment the ink was composed of 5-15 (w/w)% PEGDA3400, 6-9 (w/w)% PEGDMA 575, 1-3 (w/w)% Lithium phenyl-2,4,6-trimethylbenzoylphosphinate and 0.13 (w/w)% UV386A and mixed speed mixer at 200 RPM for 3 min.

The ink was poured in to the vat of 3D printer. The printer was custom made and designed for hydrogel 3D printing. Embodiments of the systems and methods used have been discussed in application 63/069317 filed Aug. 24, 2020 which is incorporated by reference. The microfluidic was printed by photopolymerized ink. The bioink was printed by layer by layer photopolymerization method. The architecture of the printed scaffold was capsule-net with the dimension of 4 mm×3 mm×14 mm

The scaffold was seeded with various cells including Pulmonary Artery Endothelial Cells (PAEC) in the vasculature side and Pulmonary alveolar epithelial cells in the airway side (ATCC cell lines, Manassas, Va., USA). The cells were seeded at the flow rate of 30 ul/min for 6 hours following by perfusion of buffer for 4 days.

The scaffold was tested by perfusing blood from one side and air from another side. Deoxygentated blood with 50% SpO₂ level was perfused from one side and the amount of oxygenation was recorded from another side. The flow rate of the blood was set to 200 ul/min and blood was collected before and after passing to the microphysiological system. The level of gases in the blood were measured using a Radiometer Blood analyzer.

Example 2:

Gas exchange units were generated using the 3-D printing techniques described herein. A capsule net architecture was made out of 602N material with dimensions of 20 um and 385 nm. The generated chip was placed inside of an incubator at 37 C. 100 mL of horse blood secured and brought into a lower oxygen content of about SpO₂ of about 60% using an oxygenator/deoxygenator. The vasculature component of the chip was then infused with the horse blood and either nitrogen or air was flowed through the airway of the chip.

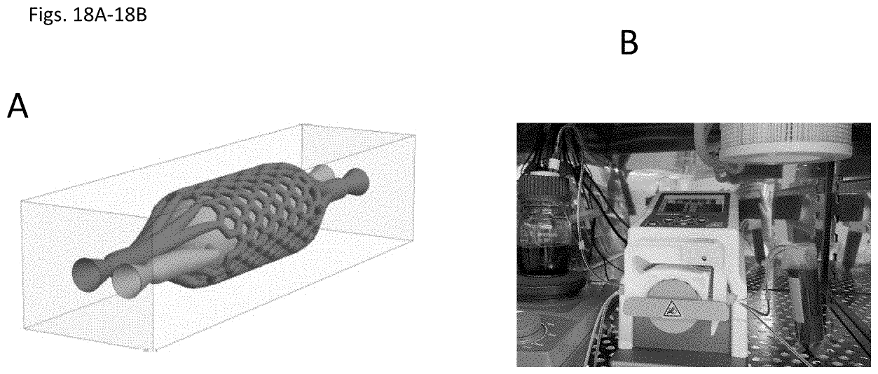

FIG. 18A shows the capsule net architecture design of the gas exchange unit used in the samples of Example 2.

FIG. 18B shows an image of the setup of the temperature controlled gas exchange unit with blood inlet and outlet for Chips 1-5.

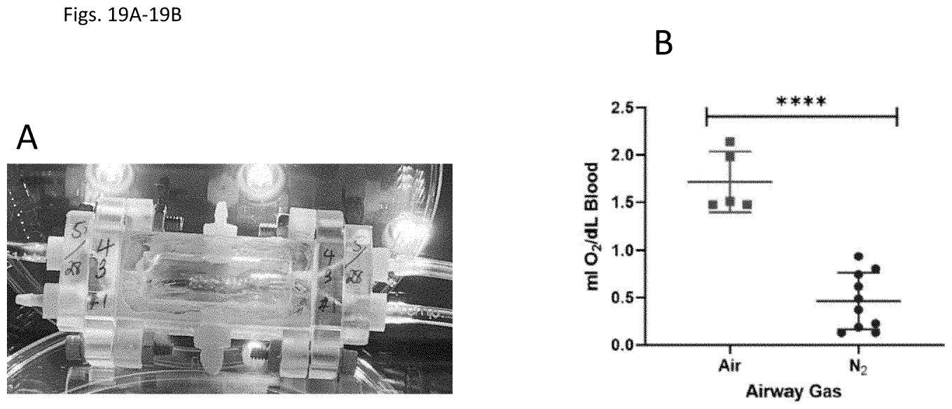

FIG. 19A shows an image of the Chip 1 setup. The air/nitrogen inlet was attached with 0.5 psi of pressure and a gas flow rate of 200 ul/min was used for perfusion of the chip. FIG. 19B shows the measurement comparison of the blood oxygen content of the blood at the outlet of the chip when air is flowed through the airways vs. nitrogen is flowed through the airway. As shown, the gas transfer for both air and nitrogen was significant.

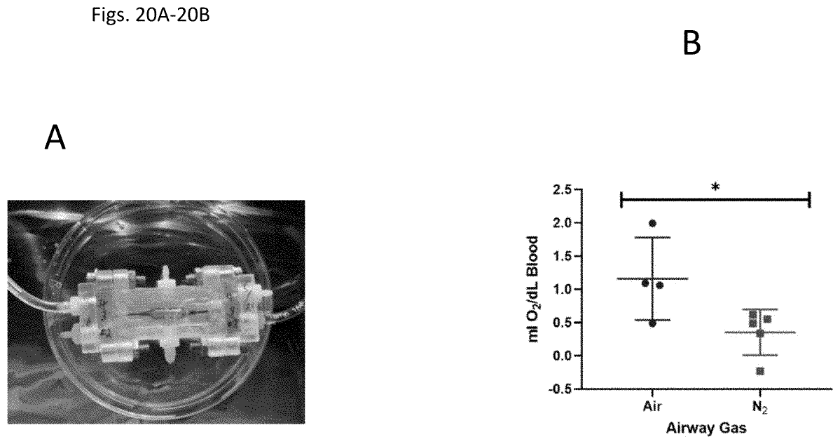

FIG. 20A shows an image of the Chip 2 setup printed and setup in the same manner as chip 1. FIG. 20B shows the measurement comparison of the blood oxygen content of the blood at the outlet of the chip when air is flowed through the airways vs. nitrogen is flowed through the airway. As shown, the gas transfer for both air and nitrogen was significant. There was some batch variation between chips 1 and 2.

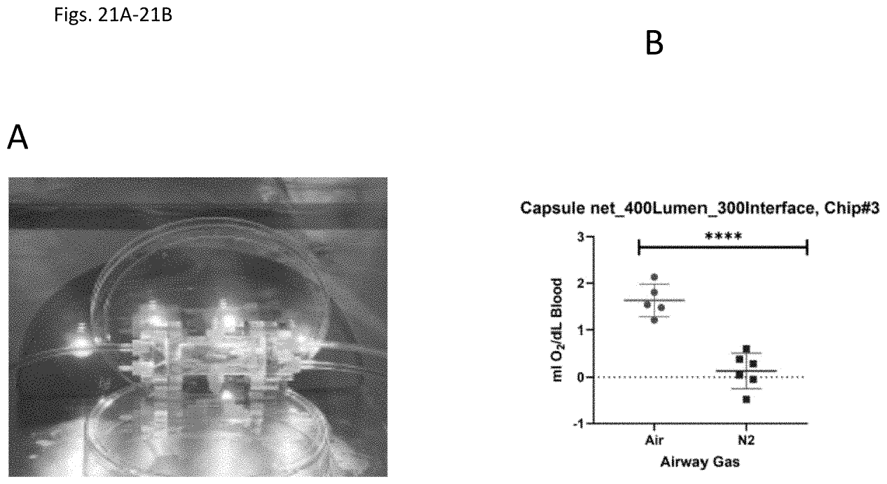

FIG. 21A shows an image of the Chip 3 setup in the same manner as chip 1. FIG. 21B shows the measurement comparison of the blood oxygen content of the blood at the outlet of the chip when air is flowed through the airways vs. nitrogen is flowed through the airway. The gas exchange results for both air and nitrogen are significant. The results for the air part is in very good agreement with the chip number 1 with similar conditions.

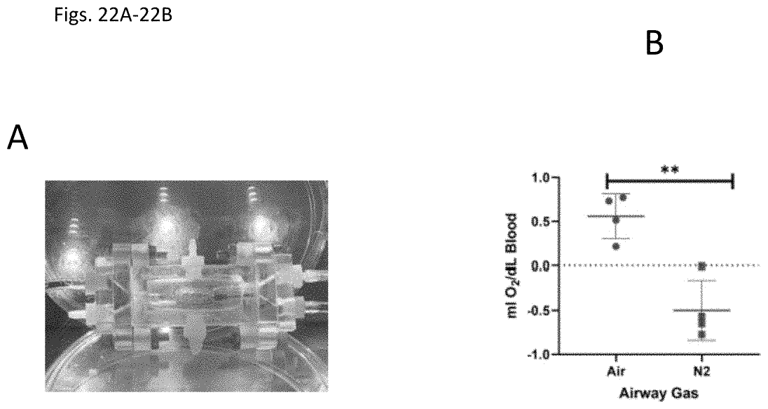

FIG. 22A shows an image of the Chip 4 setup in the same manner as Chip 1. FIG. 22B shows the measurement comparison of the blood oxygen content of the blood at the outlet of the chip when air is flowed through the airways vs. nitrogen is flowed through the airway. As shown, the gas transfer for both air and nitrogen was again significant.

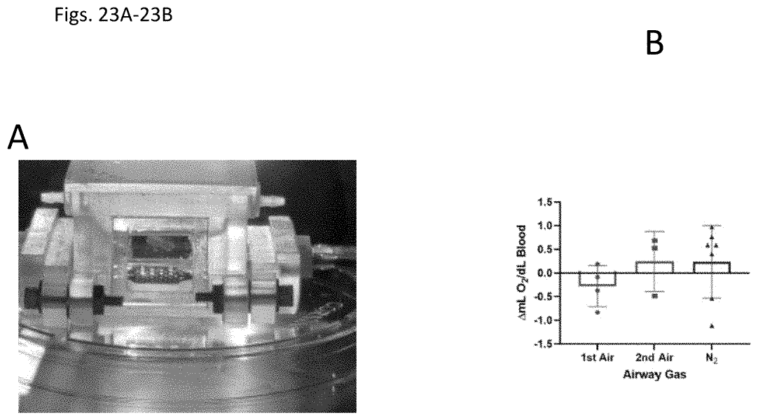

FIG. 23A shows an image of the Chip 5 setup. Chip 5 was set up in a similar manner to Chip 1, however, a higher flow rate of 400 ul/min was used for perfusion of the chip. FIG. 23B shows the measurement comparison of the blood oxygen content of the blood at the outlet of the chip when air is flowed through the airways vs. nitrogen is flowed through the airway. The higher flow rate of 400 uL/min resulted in no meaningful gas transfer for oxygen and nitrogen compared to those with no ventilation and performed much worse than the samples at 200 uL/min. This is likely due to the blood not having enough time for the diffusion transfer.

While preferred embodiments have been illustrated and described, it should be understood that changes and modifications can be made therein in accordance with ordinary skill in the art without departing from the invention in its broader aspects as defined herein.

All references disclosed herein are specifically incorporated by reference thereto.

1. A microphysiological system for evaluating 3D printed scaffold and cell interaction, the system comprising:
a. a 3D printed microfluidic hydrogel holder printed as a plastic part which is configured to hold a microfluidic hydrogel for microscopic imaging; and
b. a 3D printed microfluidic hydrogel which has a vasculature structure compartment and an airway compartment.

2. (canceled)

3. A microphysiological unit comprising:
(a) a vascular network configured to conduct a fluid; and
(b) an airway compartment configured to hold a gas,
wherein the vascular network contacts the airway compartment to permit gas exchange between the gas and fluid, and wherein each of the vascular network and the airway compartment comprises a polymer scaffold.

4. The microphysiological unit of claim 3, wherein the fluid is blood.

5. The microphysiological unit of claim 3, wherein the gas comprises oxygen.

6. (canceled)

7. The microphysiological unit of claim 3, wherein the polymer scaffold of the vascular network is seeded with pulmonary artery endothelial cells.

8. The microphysiological unit of claim 3, wherein the polymer scaffold of each of the vascular network and the airway compartment is a hydrogel scaffold.

9. The microphysiological unit of claim 3, wherein the polymer scaffold of the vascular network and the polymer scaffold of the airway compartment comprise different monomers.

10. The microphysiological unit of claim 3, wherein the polymer scaffold of each of the vascular network and the airway compartment comprises one or more compounds selected from the group consisting of polyethylene glycol, polyethylene glycol diacrylate, polyethylene glycol methacrylate, polyethyleneglycolmethylether, N,N′-methylenebiasacrylamide and methacrylated collagen.

11. The microphysiological unit of claim 3, wherein a diameter of an interface between the vascular network and the airway compartment is between 250 microns and 350 microns.

12. The microphysiological unit of claim 3, wherein a diameter of a lumen of the vascular network is between 350 microns and 450 microns.

13. (canceled)

14. The microphysiological unit of claim 3, wherein a diameter of a lumen of the vascular network is greater or equal to a diameter of an interface between the vascular network and the airway compartment.

15. An artificial lung comprising a cellularized or acellular gas exchange unit, which is the microphysiological unit of claim 3.

16. A method of forming a gas exchange unit, comprising printing a gas exchange unit comprising a vascular network configured to conduct a fluid and an airway compartment configured to hold a gas, wherein the vascular network contacts the airway compartment to permit gas exchange between the fluid and the gas.

24. A microphysiological unit comprising (a) a vascular network configured to allow a complex vasculature structure to cellularize using endothelial cells to form a vasculature tissue and to conduct blood and (b) an airway compartment configured to hold air and to cellularize with epithelial cells, wherein the vascular network contacts the airway compartment to permit gas exchange.

25. A method of screening a pharmaceutical composition utilizing the microphysiological unit of claim 24.

26. A method of modeling a pulmonary disorder utilizing the microphysiological unit of claim 24.

27. A method of performing a pulmonary toxicity study utilizing the microphysiological unit of claim 24.

28-30. (canceled)

31. The microphysiological unit of claim 3 comprising a 3D printed biomaterial hydrogel scaffold at human organ scale.

32. (canceled)

33. The microphysiological unit of claim 31, wherein the 3D printed biomaterial hydrogel scaffold comprises one of Collagen and Gelatin.

34. The microphysiological unit of claim 31, which is seeded with cells.

35. The microphysiological unit of claim 34, wherein the cells are endothelial, epithelial, fibroblast, smooth muscle cells.

36. The microphysiological unit of claim 31, wherein small airway epithelial cells (SAEC) are seeded on one side of the biomaterial hydrogel scaffold and endothelial cells are seeded on the other side of the biomaterial hydrogel scaffold.

37. (canceled)
